(19) United States
(12) Patent Application Publication
(10) Pub. No.: US 2006/0015959 A1
(43) Pub. Date: Jan. 19, 2006

(54) Transgenic animals expressing transglutaminase II

(75) Inventors: Feng Bian, Ann Arbor, MI (US); Rathna Iyer, Novi, MI (US); Kevin K. Wang, Gainesville, FL (US); Yuang Ye, Ann Arbor, MI (US)

Correspondence Address: James A. Jubinsky;5th Floor, 150 East 42nd Street, New York, NY, 10017, US

(73) Assignee: Warner-Lambert Company

(21) Appl. No.: 11/167,991

(22) Filed: Jun. 27, 2005

Related U.S. Application Data
(60) Provisional application No. 60/583,415, filed on Jun. 28, 2004.

(51) Int. Cl. A01K 67/027; C07H 21/04; C12N 5/06

(52) U.S. Cl. 800/18; 435/354; 435/320.1; 536/23.2

(57) ABSTRACT

The invention provides transgenic, non-human animals and transgenic non-human mammalian cells harboring a transgene encoding a TGII (activator of the protein kinase cdk 5) polypeptide. The two neuropathological lesions associated with Alzheimer's disease (AD) are amyloid plaques and neurofibrillary tangles (NFTs), composed predominantly of amyloid β peptides and hyperphosphorylated tau, respectively. While animal models for plaque formation exist, there is no animal model that recapitulates the formation of NFTs. This invention provides transgenic mice that overexpress human TGII, an activator of cdk5, resulting in tau that is hyperphosphorylated at AD-relevant epitopes. Deposition of tau is detected in the amygdala, thalamus and cortex. Increased phosphorylated neurofilament, silver-positive neurons and neuronal death are also observed in these regions. We conclude that the overexpression of TGII, an activator of cdk5, is sufficient to produce hyperphosphorylation of tau and neuronal death. The TGII transgenic mouse represents the first model for tau pathology in AD.

This application claims priority under 35 U.S.C 119 of U.S. Provisional 60/583,415 filed Jul. 28, 2004. The entire contents of the prior application are incorporated herein by reference.

### TECHNICAL FIELD

The invention provides transgenic, non-human animals and transgenic non-human mammalian cells harboring a transgene encoding a Transglutaminase II (TGII) polypeptide. The invention also provides non-human animals and cells comprising a transgene encoding a TGII polypeptide and further comprising functional overexpression of TGU, the TGII transgene and targeting constructs used to produce such transgenic cells and animals, transgenes encoding human TGII polypeptide sequences and methods for using the transgenic animals in pharmaceutical screening and as commercial research tools for modeling neurodegenerative disease such as Alzheimer's disease and TGII/tau and/or TGII/amyloid beta peptide biochemistry in vivo.

### BACKGROUND OF THE INVENTION

Throughout the specification, a number of publications are cited. These publications are hereby incorporated herein by reference in their entirety.

Alzheimer's disease (AD) is a progressive, neurodegenerative disorder characterized by loss of cognitive function. The primary neuropathological lesions in AD are amyloid plaques and neurofibrillary tangles (NFTs). Amyloid plaques are composed primarily of amyloid beta (Aβ) peptides, varying in length from 39-42 amino acids, which are derived from amyloid precursor protein (APP). For example, see Selkoe D J (1998) Trends in Cell Biology 8: 447-53; and Hardy J, Selkoe D J (2002) Science 297: 353-356. Aβ

NFTs are composed of the microtubule binding protein tau that is hyperphosphorylated at epitopes which exist in a predominantly unphosphorylated state in disease-free brain. For example, see Goedert, M. (1997). “The Neurofibrillary Pathology of Alzheimer's Disease”, The Neuroscientist 3(2): 131 -141; Goedert, M., R. A. and Crowther, et al. (1998) “Tau mutations cause frontotemporal dementias” Neuron 21(5): 955-958; Spillantini, M. G. and Goedert M. (1998). “Tau protein pathology in neurodegenerative diseases.” Trends in Neurosciences 21(10): 428-433. The respective roles that these lesions play in the neuronal loss and dementia observed in patients with AD remain controversial.

Thus, amyloid beta peptides including Aβ40, Aβ42, Aβ11-42, and Aβ11-40; and tau protein form insoluble aggregates in AD, leading to amyloid plaques and neurofibrillary tangles, respectively. TGII is induced and activated in Alzheimer's disease and Huntington's Disease. For example, see Lesort et al. (2000) Prog Neurobiol 61(5): 439-63; Selkoe, D J (2002) Neurochem Int 40(1): 13-6; Singer, S. M., G. M. Zainelli, et al. (2002) Neurochem Int 40(1): 17-30. Strong in vitro and in vivo evidence shows that TgII contributes to the formation and/or stability of aggregated proteins by intermolecular covalent crosslinking (between side chains of lysine and glutamine residues on both tau and Aβ) (Zhang, W. et al. (1998) Acta Neuropathol (Berl). 96(4): 395-400; Singer, S. M., G. M. Zainelli, et al. (2002) Neurochem Int 40(1): 17-30).

Thus, since both amyloid plaques and neurofibrillary tangle formation are strongly linked to the pathology of AD, it is envisioned that TgII inhibitors would constitute new treatments for AD. Therefore, transgenic animals overexpressing TGII are useful as a tool for screening TgII inhibitors.

TgII-overexpressing transgenic mice are also useful as crossing intermediates for crossing with huntingtin-, APP- and tau-transgenic mice, and for in vivo validation of TgII as a target for Alzheimer's disease. In addition, crossing TgII-transgenic mice and huntingtin-transgenic mouse models of Huntington's disease may accelerate phenotypic development of the disease model and provide a rationale for TgII inhibition as a treatment for Huntington's disease. For example, see Karpuj, M V et al. (2002) Nat Med 8(2): 143-9; Karpuj M VBecher, et al. (2002) Neurochem Int 40(1):31-6.

The development of experimental models of Alzheimer's disease that can be used to define further the underlying biochemical events involved in AD pathogenesis is highly desirable. Such models could be employed, in one application, to screen for agents that alter the degenerative course of AD. For example, a model system of AD could be used to screen for environmental factors that induce or accelerate the pathogenesis of AD. Alternatively, an experimental model could be used to screen for agents that inhibit, prevent, or reverse the progression of AD. Such models could be employed to develop pharmaceuticals that are effective in preventing, arresting or reversing AD. Only humans and aged non-human primates develop any of the pathological features of AD. The expense and difficulty of using primates and the length of time required for developing the AD pathology makes extensive research on such animals prohibitive. Rodents do not develop AD, even at an extreme age.

Based on the above, it is clear that a need exists for nonhuman cells and nonhuman animals which exhibit higher expression of TGII. These transgenic TGII mice are useful for crossing with known animal models of disease, including those models of Huntington's disease and other trinucleotide repeat disorders, Alzheimer's disease (APP transgenics, presenilin transgenics, and tau transgenics), the tauopathies (tau transgenics and p25 transgenics), Parkinson's disease (alpha synuclein transgenics, celiac disease; and accelerating the development of the desired pathology.

Thus, it is an object of the invention herein to provide methods and compositions for transferring transgenes and homologous recombination constructs into mammalian cells, especially into embryonic stem cells. It is also an object of the invention to provide transgenic non-human cells and transgenic nonhuman animals harboring transgenes resulting in the increased expression of TGII, a cross-linker of of Aβ peptides, and tau proteins. Of further interest to the present invention are the application of such transgenic animals as in vivo systems for screening test compounds for the ability to decrease or eliminate the activity of TGII, in turn leading to prevention, decrease or elimination of cross-linked Aβ peptide(s) and tau protein, and the associated amyloid plaques and neurofibrillary tangles.

It is desirable to provide methods and systems for screening test compounds for the ability to inhibit or prevent the cross-linking of tau and associated amyloid neurofibrillary tangle formation. It is desirable to provide methods and systems for screening test compounds for the ability to inhibit or prevent the cross-linking of Aβ peptide(s) and associated amyloid plaque formation. In particular, it is be desirable to base such methods and systems on inhibition of TGII, where the test compound blocks cross-linking of tau and/or Aβ peptide(s) mediated by TGII, and the test compound also blocks the respective tangle and/or plaque formation. Such methods and transgenic animals should provide a rapid, economical and suitable way for screening large numbers of test compounds.

### SUMMARY OF THE INVENTION

In one embodiment, the present invention is directed to recombinant DNA comprising a mouse Thy1.2 promoter operably linked to a human TGII polypeptide encoding sequence.

In a preferred embodiment, the present invention is directed to recombinant DNA wherein said sequence encoding said TGII is genomic DNA.

In another preferred embodiment, the present invention is directed to recombinant DNA wherein said sequence encoding said TGII polypeptide is cDNA.

In still another preferred embodiment, the present invention is directed to recombinant DNA wherein said sequence is that of SEQ ID NO: 1.

In yet another embodiment, the present invention is directed to a vector comprising recombinant DNA according to the present invention.

In another embodiment, the present invention is directed to eukaryotic cell lines comprising recombinant DNA according to the present invention.

In another embodiment, the present invention is directed to a transgenic non-human animal, or progeny thereof, whose germ cells and somatic cells express recombinant DNA according to the present invention.

In a preferred embodiment, the present invention is directed to a transgenic non-human animal, or progeny thereof, which is a mouse.

In a further embodiment, the present invention is directed to a method for treating an animal having a disease characterized by the increased expression of a TGII polypeptide comprising administering a therapeutically effective amount of an inhibitor of expression of said TGII polypeptide.

In another embodiment, the present invention is directed to a method for determining the ability of a compound to inhibit the expression of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression said TGII polypeptide by said compound.

In still another embodiment, the present invention is directed to a method for generating data to determining the ability of a compound to inhibit the expression of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression of said TGII polypeptide by said compound.
e using the data derived from said inhibition to synthesize compounds capable of inhibiting the expression of said TGII fragment.

In a further embodiment, the present invention is directed to a method for treating an animal having a disease characterized by the increased expression of a TGII polypeptide comprising administering a therapeutically effective amount of an inhibitor of cross-linking activity of said TGII polypeptide.

In another embodiment, the present invention is directed to a method for determining the ability of a compound to inhibit the cross-linking activity of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression of said TGII polypeptide by said compound.

In still another embodiment, the present invention is directed to a method for generating data to determining the ability of a compound to inhibit the cross-linking activity of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression of said TGII polypeptide by said compound.
e. using the data derived from said inhibition to synthesize compounds capable of inhibiting the cross-linking activity of said TGII fragment.

### BRIEF DESCRIPTION OF THE FIGURES

FIG. 1 depicts a schematic representation of constructs used for making mice overexpressing TGII in a neuron-specific manner.

FIG. 2 depicts expression levels of full length human TGII polypeptide in different lines of transgenic but not wildtype mice (Ctx=Cortex; Cbm=cerebellum).

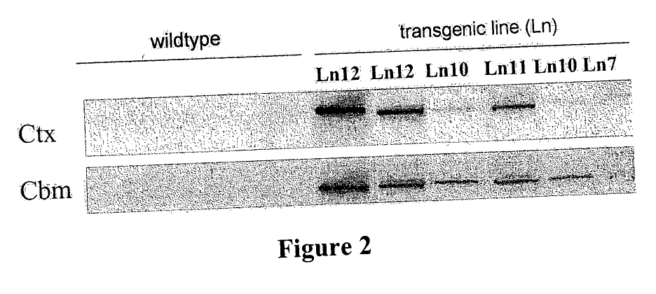

FIG. 3 depicts higher enzymatic (cross-linking) activity in brain lystaes of transgenic mice v. those of wildtype mice.

### DETAILED DESCRIPTION

Generally, the nomenclature used herein and the laboratory procedures in cell culture, molecular genetics, nucleic acid chemistry and hybridization, biochemistry, histology and immunocytochemistry described below are those well known and commonly described in the art. Standard techniques are used for recombinant nucleic acid methods, polynucleotide synthesis, cell culture, transgene incorporation, Western blotting, immunocytochemistry and histological techniques such as silver staining. The techniques and procedures are generally performed according to conventional methods in the art and various general references which are provided throughout this specification. The procedures therein are well known in the art and are provided for the convenience of the reader. All the information contained therein is incorporated herein by reference.

In accordance with the foregoing objects, in one aspect of the invention are provided nonhuman animals harboring at least one copy of a transgene comprising a polynucleotide sequence that encodes a heterologous protein operably linked to regulatory elements that are capable of expressing the heterologous protein in the transgenic nonhuman animal. Said heterologous polypeptide is a full length human transglutaminase II (TGII; Accession NM_{—}004613; Gentile V et al. (1994) Genomics 20(2), 295-297; SEQ ID NO: 2). Typically, the nonhuman transgenic animal is a mouse and the heterologous gene is the human TGII sequence. Transgenes are typically cDNA sequences that have been operably linked to cis-acting regulatory sequences that direct expression in the host transgenic mammal in cell-type specific manner, typically in neurons. Typically, the transgene will be incorporated into the host chromosomes in random, non-targeted fashion. The invention further provides that the nonhuman, transgenic animal harboring at least one copy of the heterologous TGII sequence transgene or gene targeting vector of the invention either non-homologously or homologously integrated into the chromosomal location express the TGII polypeptide. Transgenic animals are typically produced by introduction of a transgene by microinjection into pronuclei of one-cell embryos or a targeting vector into the host by electroporation, lipofection, or viral transfection of embryonic stem (ES) cells. The transgenic animals that express the TGII polypeptide are suitable for use as models of disease and screening of potential therapeutic compounds including small molecules, proteins and polynucleotides. The invention also provides nonhuman or human cell lines to be derived by transformation of established cell lines or primary cell lines established directly from the nonhuman transgenic animal, for example neurons. It is recognized that the transgenic nonhuman animal can have additional genetic modifications by transgenic art or through traditional matings with other transgenic or naturally occurring animals to produce novel animals that serve as alternative disease models, drug screens or other applications.

This includes the inactivation of the murine endogenous p25 gene through gene targeting in ES cells and resultant murine p25 mice that becomes the preferred host for expression of the human TGII heterologous polypeptide, a cross-linker of Aβ peptides and Tau proteins. Such heterologous transgenes may be integrated in nonhomologous chromosomal locations in the transgenic animal, typically derived by pronuclear injection or may be integrated by gene targeting in ES cells by methods to inactivate the murine p25 gene and add linked sequences to direct expression of the heterologous human TGII sequences.

In general, the invention encompasses methods for the generation and characterization of transgenic animals that express the human TGII polypeptide, a cross-linker of Aβ peptides and Tau proteins. The transgenic animals express this human protein in the presence of the endogenous homologue. The techniques and procedures are performed according to established protocols that are generally considered to be routine methods in the art and references are provided within this specification. The transgenic mice are thus useful to establish the role of the human TGII in the formation of cross-linked tau, Aβ peptides, and/or otherpathogenic proteins in neurodegenerative conditions including Alzheimer's disease, Down's syndrome, Cerebral Aβ Angiopathy, Multiple system Taupathy (familial), Progressive Supranuclear Palsy, neurofilamentopathies, Corticobasal Degeneration, Pick's Disease, Diffuse Lewy Body Disease, Parkinson's disease, MultipleSystem Atrophy, Amyotrophic Lateral Sclerosis (ALS), Familial ALS, Triplet Repeat Disorders including HD and the like, Huntington's chorea, Prion Diseases including CJD and the like, Familial British Dementia, Familial Danish Dementia, Familial Encephalopathy with Neuroserpin Inclusion Bodies (FENIB), Familial Cerebral Hemorrhage with Amyloidosis (Icelandic), Familial Amyloidotic Neuropathy, Stroke, Head Trauma, and other neurodegenerative diseases; and in other pathological conditions including CADASIL (Notch -3), Cataract of Lens, and Celiac disease (Mowat, A.M. (2003) Lancet 361(9365): 1290-2. Such transgenic animals can be commercially marketed to researchers, among other uses.

It is apparent that the preparation of other transgenic animals that express the human TGII protein is easily accomplished including rats, hamsters, guinea pigs and rabbits. The transgenic animals that express the human TGII protein can be monitored for the level of expression, and tau and Aβ cross-linking. It will be appreciated that under different conditions the level of expression and degree of tau and Aβ cross-linking will be inhibited in such an animal model. In particular, the screening of therapeutic agents that inhibit the activity of the human TGII protein will be greatly facilitated in animal models. It is apparent that the development of cell lines from the affected transgenic animals, (e.g., neurons) will improve the throughput in which biochemical and pharmacological analysis of the human TGII protein can be assessed.

Particularly preferred animal models for TGII overexpression are transgenic mammals which express TGII as described above. Such transgenic animals, particularly transgenic mice according to this invention, produce high quantities of TGII, and cross-linked tau and Aβ which can be detected according to the methods of the present invention. In accordance to this invention particular, the overexpression of TGII will be equal to or greater than endogenous TGII expression in such animals. Further, this level of TGII overexpression results in tau and/or Aβ peptide(s) cross-linking which is minimal in wildtype animals. With such elevated levels of TGII, monitoring of tau and/or Aβ peptide(s) cross-linking is greatly facilitated. In particular, screening for compounds and other therapies for inhibiting tau and/or Aβ peptide(s) cross-linking are greatly simplified in animals overexpressing TGII according to this invention.

Agents are administered to test animals, such as test mice, which are transgenic and which overexpress TGII. Particular techniques for producing transgenic mice which overexpress TGII are described below. It will be appreciated that the preparation of other transgenic animals overexpressing TGII may easily be accomplished, including rats, hamsters, guinea pigs, rabbits, and the like. In light of this disclosure, the effect of test compounds on the tau and/or Aβ peptide(s) cross-linking in the test animals may be measured in various specimens from the test animals.

The effect of test agents on tau and/or Aβ peptide(s) cross-linking may be measured in various specimens from the test animals. In all cases, it will be necessary to obtain a control value which is characteristic of the level of tau and/or Aβ peptide(s) cross-linking in the test animal in the absence of the test compound(s). In cases where the animal is sacrificed, it will be necessary to base such control values on an average or a typical value from other test animals which have been transgenically modified to overexpress TGII but which have not received the administration of any test compounds or any other substances expected to affect the level of tau and/or Aβ peptide(s) cross-linking. Once such control level is determined, test compounds can be administered to additonal test animals, where deviation from the average control value indicates that the test compound had an effect on the tau and/or Aβ peptide(s) cross-linking in the animal. Test substances considered positive, i.e., likely to be beneficial in the treament of AD or other neurodegenerative diseases, will be those which are able to reduce the level of tau phosphorylation or neuronal death preferably by at least 20% and most preferably by 80%. In addition there may be paired helical or straight filament formation in transgenic animals which overexpress TGII, and display tau and/or Aβ peptide(s) cross-linking. In these cases, test compounds can be administered to test animals and the reduction of filament formation monitored as a result of exposure to the compound. In addition, there may be behavioral alterations in the transgenic animals which overexpress TGII and display tau tau and/or Aβ peptide(s) cross-linking. In these cases it will be necessary to obtain a control value from live animals performing a behavioral task (e.g., the measurement of locomotor activity) in the test animal in the absence of test compound(s). Such a control will also be determined in non-transgenic, wild type mice. The difference between the wild type and transgenic mice will serve as the outcome measure for the effects of compounds. Once such control levels are determined, test compounds can be administered to additional test animals, where reduction in or reversal of the difference between the wild type and transgenic mice indicates that test compound has an effect on the behavioral test being measured. Test substances considered positive, i.e., likely to be beneficial in the treament of AD or other neurodegenerative diseases, preferably will be those which are able to reverse or, substantially reverse, or favorably modify the behavioral abnormality in the transgenic animal to the level found in wild type mice.

Test agents will be defined as any small molecule, protein, polysaccharides, deoxy or ribomucleotides, or any combination thereof that when added to the cell culture or animal will not adversely interfere with the cell or animal viability. Agents that alter the level of human TGII expression, and tau and/or Aβ peptide(s) cross-linking will be considered as candidates for further evaluation as potential therapeutics. The test compound will typically be administered to transgenic animals at a dosage of from 1 ng/kg to 100 mg/kg, usually from 10 ug/kg to 32 mg/kg.

Test compounds which are able to inhibit tau and/or Aβ peptide(s) cross-linking are considered as candidates for further determinations of the ability to block tau and/or Aβ peptide(s) cross-linking in animals and humans. Inhibition of tau and/or Aβ peptide(s) cross-linking indicates that TGII activity has been at least partly blocked, reducing the amount of TGII available to cross-link tau and/or Aβ peptide(s).

The present invention further comprises pharmaceutical compositions incorporating a compound selected by the above-described method and including a pharmaceutically acceptable carrier. Such pharmaceutical compositions should contain a therapeutic or prophylactic amount of at least one compound identified by the method of the present invention. The pharmaceutically acceptable carrier can be any compatible, non-toxic substance suitable to deliver the compound or compounds to an intended host. Sterile water, alcohol, fats, waxes and inert solids may be used as the carrier. Pharmaceutically acceptable adjuvants, buffering agents, dispersing agents, and the like may also be incorporated into the pharmaceutical compositions. Preparation of pharmaceutical compositions incorporating active agents is well described in the medical and scientific literature. See, for example, Remington's Pharmaceutical Sciences, Mack Publishing Company, Easton, Pa., 16^{th} Ed., 1982, the disclosure of which is incorporated herein by reference.

The pharmaceutical compositions just described are suitable for systemic administration to the host, including both parenteral, topical, and oral administration. The pharmaceutical compositions may be administered parenterally, i.e., subcutaneously, intramuscularly or intravenously. Thus, the present invention provides compositions for administration to a host where the compositions comprise a pharmaceutically acceptable solution of the identified compound in an acceptable carrier, as described above.

Commercial Research and Screening Uses

Non-human animals comprising transgenes which encode TGII can be used commercially to screen for agents having the effect of preventing or reducing the cross-linking of tau and/or Aβ peptide(s). Such agents can be developed as pharmaceuticals for treating tau and/or Aβ peptide cross-linking, AD, and other neurodegenerative diseases. For example, the p53 knockout mice of Donehower et al. (54) have found wide acceptance as commercial prouducts for carcinogen screening and the like. The transgenic animals of the present invention exhibit abnormal tau and/or Aβ peptide cross-linking and can be used for pharmaceutical screening and as disease models for neurodegenerative and other diseases involving TGII/tau and/or Aβ peptide biochemistry. Such animals have many uses including but not limited to identifying compounds that affect tau and/or Aβ peptide cross-linking; in one variation, the agents are thereby identified as candidate pharmaceutical agents. The transgenic animals can also be used to develop agents that modulate TGII expression and or stability; such agents can serve as therapeutic agents to treat neurodenerative diseases.

The TGII overexpressing mice of the invention can also serve as disease models for investigating tau-related pathologies (e.g., AD, Pick's disease, Parkinson's disease, frontal temporal lobe dementia associated with chromosome 17, stroke, traumatic brain injury, mild cognitive impairment and the like).

Other features and advantages of the invention will be apparent from the following detailed description and from the claims. While the invention is described in connection with specific embodiments, it will be understood that other changes and modifications that may be practiced are also part of this invention and are also within the scope of the appendant claims. This application is intended to cover any equivalents, variations, uses, or adaptations of the invention that follow, in general, the principles of the invention, including departures from the present disclosure that come within known or customary practice within the art, and that are able to be ascertained without undue experimentation. Additional guidance with respect to making and using nucleic acids and polypeptides is found in standard textbooks of molecular biology, protein science, and immunology (see, e.g., Davis et al., Basic Methods in Molecular Biology, Elsevir Sciences Publishing, Inc., New York, N.Y.,1986; Hames et al., Nucleic Acid Hybridization, IL Press, 1985; Molecular Cloning, Sambrook et al., Current Protocols in Molecular Biology, Eds. Ausubel et al., John Wiley and Sons; Current Protocols in Human Genetics, Eds. Dracopoli et al., John Wiley and Sons; Current Protocols in Protein Science, Eds. John E. Coligan et al., John Wiley and Sons; and Current Protocols in Immunology, Eds. John E. Coligan et al., John Wiley and Sons). All publications, including published patent applications and issued patents, mentioned herein are incorporated by reference in their entireties. Having described the invention in general terms, reference is now made to specific examples. It is to be understood that these examples are not meant to limit the present invention, the scope of which is to be determined by the appended claims.

### EXPERIMENTAL EXAMPLES

Methods

Preparation of Lysate

Frozen brain tissue was powdered with pre-cooled mortar and pestle over dry ice. Approximately 50 mg of powdered tissue was resuspended in Triton lysis buffer [1% Triton, 20 mM Tris-HCl, 150 mM NaCl, 5 mM EDTA, 5 mM EGTA, 1 mM DTT]. The samples were placed on ice for 15 minutes with intermittent vortexing. The lysate was cleared by centrifugation at 20,000× g for 15 min at 4° C. Protein concentration in the cleared lysate was determined using a modified Lowry assay (Bio-Rad). Samples were stored in 50% glycerol at −20° C. until use.

Transglutaminase Activity Assay

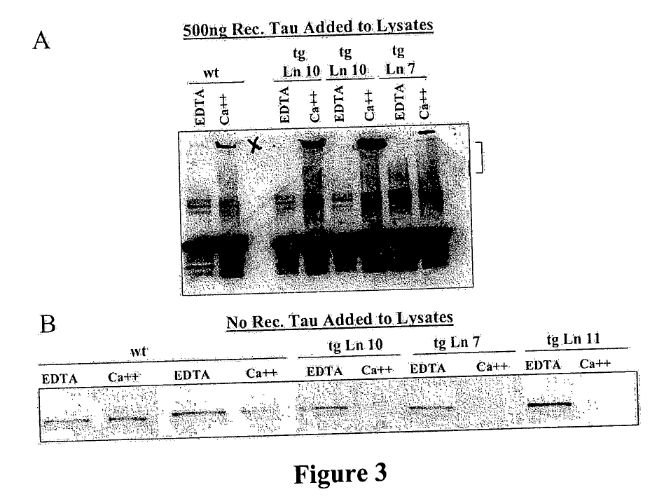

15 μL of brain lysate was diluted in a buffer containing 1 M HEPES (pH7.4) and 1 M DTT with or without 20 mM CaCl₂ and with our without 50 ng of recombinant Tau (Panvera) (as indicated in FIG. 3) in a total volume of 30 μL. (As a positive control, brain lysates from wt animals were treated with 2 or 10 μL guinea pig transglutaminase in buffer containing 20 mM CaCl₂). After incubation of samples for 120 mins. at RT, 5 uL of 8× SDS- loading buffer was added to the samples and assessed by western blotting.

Western Blotting

Samples were separated by SDS/PAGE [4-20%(w/v) acrylamide] with a Tris/glycine running buffer system and then transferred to a nitrocellulose membrane using a semi-dry electrotransferring unit (Bio-Rad) at 20 mA for 2 h. The blots were probed with specific primary antibodies [total tau antibody (Immunogen), 1:1000 dilution, and a commercially available antibody for hTGII] overnight. To develop the blots, an HRP-linked secondary antibody and ECL detection system were used.

Construction of the Thy1-TgII Transgenes

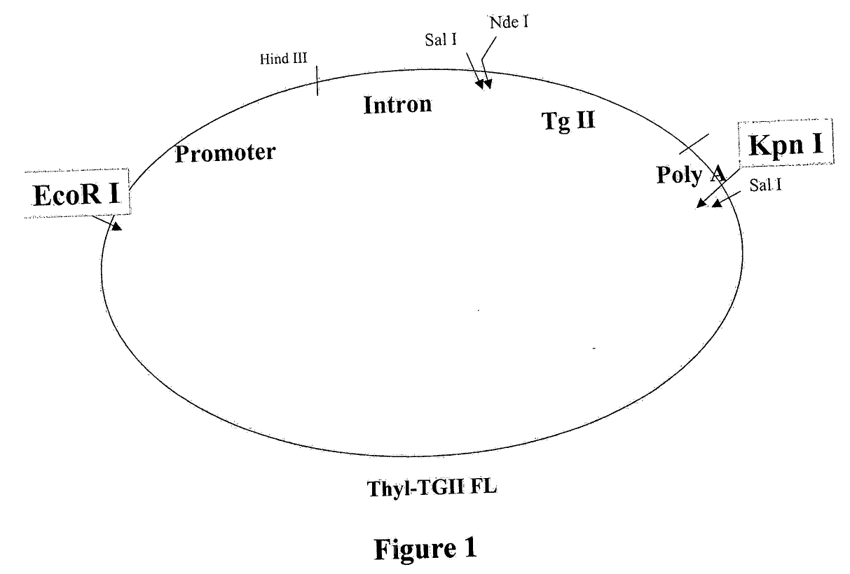

The mouse Thy1.2 promoter has been shown to express transgenes specifically in neurons (Kelley, K. A. et al. (1994) Brain Res Mol Brain Res 24(1-4): 261-74; SEQ ID NO:2). The human TGII cDNA (hTGII; SEQ ID NO:1) was cloned from a human brain cDNA library. Then, the hTGII cDNA was subcloned into PCR2.1 vector (Invitrogen Life Technologies) utilizing the primers set forth in SEQ ID NO: 3 containing a Sal I site at the 5′ end and SEQ ID NO: 4 containing 15 bp of the SV40 polyA sequence at the 3′ end. This subcloning also results in the deletion of the stop codon from the hTgII cDNA. Afterwards, the SV40 polyA sequence (SEQ ID NO:7) was attached by PCR-sewing to the 3-end of the subcloned fragment utilizing the primers set forth in SEQ ID NO's: 5 and 6. SEQ ID NO: 6 contains Kpn I and Sal I sites at the 3′ end of SV40 poly A. Following Sal I digestion, the purified 2.3 fragment containing the hTGII and SV40 polyA tail were inserted into the Sal I site of the mouse Thy1.2 promoter construct which contains Thy1.2 promoter region, exon 1, intron 1 and part of the exon 2 of the Thy1.2 gene (SEQ ID NO:2). The constructs were designated as Thy1-TGII FL (FIG. 1).

Generation of Thy1-TgII-Transgenic Mice

The above-described Thy1-TgII FL construct schematically depicted in FIG. 1 containing the Thy1.2 promoter, exon 1, intron 1 and part of the exon 2 of the Thy1.2 gene, hTGII cDNA, and SV40 Poly A tail (cumulatively about 6.5 kb) was restriction-digested with EcoR I and Kpn I to release the 6.5 kb fragment. The fragment was gel-purified and used for murine embryo microinjection.

Production of transgenic mice by pronuclear microinjection was carried out by published procedures essentially as outlined in Hogan, B. et al. (1994) Manipulating the Mouse Embryo: A Laboratory Manual, 2nd edition, Cold Spring Harbor Laboratories, New York. Pronuclear stage embryos from F1 female mice of the strain FVB/N (Charles River Labs, Wilmington, Mass.) were obtained after superovulation with 5 international units (IU) of follicle stimulating hormone from pregnant mare serum (Sigma St Louis, Mo.) and 2.5 I.U. human chorionic gonadotropin (Sigma). The actual microinjection procedure was performed as described by Wagner, T. E. et al. (1981) Proc. Natl' Acad. Sci. except that the embryos were transferred immediately to pseudopregnant CD-1 recipient females (Charles River Laboratories, Wilmington, Mass.) for development of embryos to term. Mice resulting from the reimplantation events were tested for the presence of the TGII transgene by PCR analysis of genomic DNA isolated from tail biopsies at 3 weeks of age. The mice that demonstrated positive for the presence of the TGII transgene were mated with wild type FVB/N mice (Charles River Laboratories, Wilmington, Mass.) of the opposite sex. Offspring of these matings were tested for germline transmission by PCR analysis of genomic DNA isolated from tail biopsies at 3 weeks of age. Transgenic lines were produced from founder transgenic mice and were maintained by breeding to wild type FVB/N mice and PCR genotyping for the presence of the transgene.

The experiments described below were done in mice heterozygous for the inserted transgene. These mice were derived by breeding mice positive for the transgene insert with FVB/N wild-type animals and screening offspring by PCR for presence of the TGII transgene sequences.

Results

Founders were crossed with wild-type FVB mice. F1 mice from different lines were sacrificed and brain samples were collected for western analysis using a commercially available antibody specific for hTGII (CovaLab). The data have shown that line FL2 has the highest expression level of human TGII full-length protein in their brains. Thus, the breeding of the FL12 line was continued.

It was determined that transgenic homozygosity in these mice is not lethal, at least up to the age of 3 months. The breeding between the homozygous mice was successful.

Preliminary experiments using brain lysate from TGII full-length mice suggested that full-length TGII proteins overexpressed in transgenic brains are active. In vitro experiments have shown increased, Ca2+-dependent tau aggregation when recombinant tau was added to brain lysate from mice with overexpression of hTGII (FIG. 3). Note in FIG. 3 that the increased high molecular weight total tau immunoreactive protein complex forms only when transgenic brain lysate was used. However, littermate control brain lysate does not have the same effect. When no recombinant tau was added to the brain lysate, endogenous non-aggregated tau in the TGII-overexpressing brains was significantly decreased compared to that in littermate control brains when Ca2+was added (FIG. 3). This finding suggests the occurrence of increased tau aggregation in the transgenic brains.

Additionally, 7 heterozygous FL1 2 mouse hemispheres at different ages are processed for histopathological analysis the AD silver stain, H&E, and a silver degeneration stain. The data demonstrated that the morphology of the transgenic brains was normal. There was no axonal degeneration and amyloid plaque formation in the brains of the transgenic mice.

Thus, a transgenic mouse model is created that overexpresses human full-length TGII protein in brain. Homozygous TGII-overexpressing mice are viable. Data suggests higher TGII enzyme activity in TGII-overexpressing brains.

Construction of the Thy1-TgII Transgenes

The mouse Thy1.2 promoter has been shown to express transgenes specifically in neurons (Kelley, K. A. et al. (1994) Brain Res Mol Brain Res 24(1-4): 261-74; SEQ ID NO:2). The human TGII cDNA (hTGII; SEQ ID NO:1) was cloned from a human brain cDNA library. Then, the hTGII cDNA was subcloned into PCR2.1 vector (Invitrogen Life Technologies) utilizing the primers set forth in SEQ ID NO: 3 containing a Sal I site at the 5′ end and SEQ ID NO: 4 containing 15 bp of the SV40 polyA sequence at the 3′ end. This subcloning also results in the deletion of the stop condon from the hTgll cDNA. Afterwards, the SV40 polyA sequence (SEQ ID NO: 7 ) was attached by PCR-sewing to the 3-end of the subcloned fragment utilizing the primers set forth in SEQ ID NO's: 5 and 6. SEQ ID NO: 6 contains Kpn I and Sal I sites at the 3′ end of SV40 polyA. Following Sal I digestion, the purified 2.3 fragment containing the hTGII and SV40 polyA tail were inserted into the Sal I site of the mouse Thy1.2 promoter construct which contains Thy1.2 promoter region, exon 1, intron 1 and part of the exon 2 of the Thy1.2 gene (SEQ ID NO:2). The constructs were designated as Thy1-TGII FL (FIG. 1).

Generation of Thy1-TgII-Transgenic Mice

The above-described Thy1-TgII FL construct schematically depicted in FIG. 1 containing the Thy1.2 promoter, exon 1, intron 1 and part of the exon 2 of the Thy1.2 gene, hTGII cDNA, and SV40 Poly A tail (cumulatively about 6.5 kb) was restriction-digested with EcoR I and Kpn I to release the 6.5 kb fragment. The fragment was gel-purified and used for murine embryo microinjection.

Production of transgenic mice by pronuclear microinjection was carried out by published procedures essentially as outlined in Hogan, B. et al. (1994) Manipulating the Mouse Embryo: A Laboratory Manual, 2nd edition, Cold Spring Harbor Laboratories, New York. Pronuclear stage embryos from F1 female mice of the strain FVB/N (Charles River Labs, Wilmington, Mass.) were obtained after superovulation with 5 international units (IU) of follicle stimulating hormone from pregnant mare serum (Sigma St Louis, Mo.) and 2.5 l.U. human chorionic gonadotropin (Sigma). The actual microinjection procedure was performed as described by Wagner, T. E. et al. (1981) Proc. Natl' Acad. Sci. except that the embryos were transferred immediately to pseudopregnant CD-1 recipient females (Charles River Laboratories, Wilmington, Mass.) for development of embryos to term. Mice resulting from the reimplantation events were tested for the presence of the TGII transgene by PCR analysis of genomic DNA isolated from tail biopsies at 3 weeks of age. The mice that demonstrated positive for the presence of the TGII transgene were mated with wild type FVB/N mice (Charles River Laboratories, Wilmington, Mass.) of the opposite sex. Offspring of these matings were tested for germline transmission by PCR analysis of genomic DNA isolated from tail biopsies at 3 weeks of age. Transgenic lines were produced from founder transgenic mice and were maintained by breeding to wild type FVB/N mice and PCR genotyping for the presence of the transgene.

The experiments described below were done in mice heterozygous for the inserted transgene. These mice were derived by breeding mice positive for the transgene insert with FVB/N wild-type animals and screening offspring by PCR for presence of the TGII transgene sequences.

```
SEQ ID NO: 1: Human Transglutaminase II cDNA.
atggccgaggagctggtcttagagaggtgtgatctggagctggagaccaatggccgagac
caccacacggccgacctgtgccgggagaagctggtggtgcgacggggccagcccttctgg
ctgaccctgcactttgagggccgcaactaccaggccagtgtagacagtctcaccttcagt
gtcgtgaccggcccagcccctagccaggaggccgggaccaaggcccgttttccactaaga
gatgctgtggaggagggtgactggacagccaccgtggtggaccagcaagactgcaccctc
tcgctgcagctcaccaccccggccaacgcccccatcggcctgtatcgcctcagcctggag
gcctccactggctaccagggatccagctttgtgctgggccacttcattttgctcttcaac
gcctggtgcccagcggatgctgtgtacctggactcggaagaggagcggcaggagtatgtc
ctcacccagcagggctttatctaccagggctcggccaagttcatcaagaacataccttgg
aattttgggcagtttcaagatgggatcctagacatctgcctgatccttctagatgtcaac
cccaagttcctgaagaacgccggccgtgactgctcccggcgcagcagccccgtctacgtg
ggccgggtgggtagtggcatggtcaactgcaacgatgaccagggtgtgctgctgggacgc
tgggacaacaactacggggacggcgtcagccccatgtcctggatcggcagcgtggacatc
ctgcggcgctggaagaaccacggctgccagcgcgtcaagtatggccagtgctgggtcttc
gccgccgtggcctgcacagtgctgaggtgcctaggcatccctacccgcgtcgtgaccaac
tacaactcggcccatgaccagaacagcaaccttctcatcgagtacttccgcaatgagttt
ggggagatccagggtgacaagagcgagatgatctggaacttccactgctgggtggagtcg
tggatgaccaggccggacctgcagccggggtacgagggctggcaggccctggacccaacg
ccccaggagaagagcgaaggaacgtactgctgtggcccagttccagttcgtgccatcaag
gagggcgacctgagcaccaagtacgatgcgccctttgtctttgcggaggtcaatgccgac
gtggtagactggatccagcaggacgatgggtctgtgcacaaatccatcaaccgttccctg
atcgttgggctgaagatcagcactaagagcgtgggccgagacgagcgggaggatatcacc
cacacctacaaatacccagaggggtcctcagaggagagggaggccttcacaagggcgaac
cacctgaacaaactggccgagaaggaggagacagggatggccatgcggatccgtgtgggc
cagagcatgaacatgggcagtgactttgacgtctttgcccacatcaccaacaacaccgct
gaggagtacgtctgccgcctcctgctctgtgcccgcaccgtcagctacaatgggatcttg
gggcccgagtgtggcaccaagtacctgctcaacctaaccctggagcctttctctgagaag
agcgttcctctttgcatcctctatgagaaataccgtgactgccttacggagtccaacctc
atcaaggtgcgggccctcctcgtggagccagttatcaacagctacctgctggctgagagg
gacctctacctggagaatccagaaatcaagatccggatccttggggagcccaagcagaaa
cgcaagctggtggctgaggtgtccctgcagaacccgctccctgtggccctggaaggctgc
accttcactgtggagggggccggcctgactgaggagcagaagacggtggagatcccagac
cccgtggaggcaggggaggaagttaaggtgagaatggacctcgtgccgctccacatgggc
ctccacaagctggtggtgaacttcgagagcgacaagctgaaggctgtgaagggcttccgg
aatgtcatcattggccccgcctaa
SEQ ID NO:2: THY 1.2 PROMOTER
EcoR I
gaattcagagaccgggaaccaaactagcctttaaaaaagataagtacaggagccagcaag
atggctcagtgggtaaaggtgcctaccagcaagcctgacagctgagttcagtccccacga
actacgtggtaggagaggaccaaccaactctggaaatctgttctgcaaacacatgctcac
acacacacacacaaatagtataaacaattttaaatttcatttaaaaataatttgtaaaca
aaatcattagcacaggttttagaaagagcctcttggtgacatcaagttgatgctgtagat
ggggtatcattcctgaggacccaaaaccgggtctcagcctttccccattctgagagttct
ctcttttctcagccactagctgaagagtagagtggctcagcactgggctcttgagttccc
aagtcctacaactggtcagcctgactactaaccagccatgaagaaacaaggagtggatgg
gctgagtctgctgggatgggagtggagttagtaagtggccatggatgtaatgaccccagc
aatgctggctagaaggcatgcctcctttccttgtctggagacggaacgggagggatcatc
ttgtactcacagaagggagaacattctagctggttgggccaaaatgtgcaagttcacctg
gaggtggtggtgcatgcttttaactccagtactcaggaggcagggccaggtggatctctg
tgagttcaagaccagcctgcactatggagagagttttgggacagccagagttacacagaa
aaatcctggtggaaaatctgaaagaaagagagaaagaaagaaagaaagaaaggaagaaag
aaagaaagagtggcaggcaggcaggcaggaggaaggaaggaaggaaggaaggaaggaagg
aaggaaggaaggaaggaaggaaggaaggaaggaaaataggtgcgacttcaagatccggag
ttacaagcagaatgcactgtttccctaacagggccaagtgttttgagtaactgaaggtgg
gcatgatgcctgggaagcagaaacaagccaggcagatgcaccccttgcctttgcttccga
agggctgcagtagcatggaaaagatggaaaacaaccaatccattccctttgctgatataa
caggctccaaagccaaaacctgtcactggaggctcaagagcagatctccagccaagaggc
aaaggaatgggggaagctggagggcctccctctggttatccaggcttctgaaggttcaag
caaagaaagggttacaaccttaaaaggagagcgtcccggggtatgggtagaagactgctc
caccccgacccccagggtccctaaccgtcttttccctgggcgagtcagcccaatcacagg
actgagagtgcctctttagtagcagcaagccacttcggacacccaaatggaacacctcca
gtcagccctcgccgaccaccccaccccctccatccttttccctcagcctccgattggctg
aatctagagtccctccctgctcccccctctctccccacccctggtgaaaactgcgggctt
cagcgct (Exon I starts)
gggtgcagcaactggaggcgttggcgcaccaggaggaggctgcagctaggggagtc
caggt (Intron I starts)
gagagcaggccgacgggagggacccgcacatgcaaggaccgccgcagggcgaggatgcaa
gccttccccagctacagttttgggaaaggataccagggcgctcctatatgggggcgcggg
aactggggaaagaaggtgctcccaggtcgaggtgggagaggaaggcagtgcggggtcacg
ggctttctccctgctaacggacgctttcgaagagtgggtgccggaggagaaccatgagga
aggacatcaaggacatcaaggacagcctttggtccccaagctcaaatcgctttagtggtg
cgaatagagggaggaggtgggtggcaaactggagggagcccccagcgggtgacctcgtgg
ctggctgggtgcggggcaccgcaggtaagaaaaccgcaatgttgcgggaggggactgggt
ggcaggcgcgggggaggggaaagctagaaaggatgcgagggagcggaggggggagggagc
gggagaatctcaactggtagaggaaagttaaaatgaggaaatagcatcagggtggggtta
gccaagccgggcctcagggaaaggcggcaaagtttgtctgggtgtgggcttaggtgggct
gggtatgagattcggggcgccgaaaacactgctgcgcctctgccaaatcacgctacccct
gtatctagttctgccaggcttctccagccccagccccaattcttttcttagtgttccttc
cctcccctgaatctcaagcccacactccctcctccataacccactgttatcaaatctaag
tcatttgccacccaacaaccatcagcgaggcggaagcagacgggaggagtttgagatcaa
cttgggctacatcacgagttccaggctcaccaaggcttcttaaggagaccttgtctctaa
aattaattaattaattaattaatagtcccctttctctgccacagaaccttgggatctggc
tcctggtcgcagctccccccaccccaggctgacattcactgccatagcccatccggaaat
cctagtctatttccccatggatcttgaactgcagagagaatggcagagtggcccgccctg
tgcaaaggatgttcctagcctaggtggagctcgcgaactcgcagactgtgcctctcttgg
gcaaggacaggctagacagcctgccggtgtgttgagctagggcactgtggggaaggcaga
gaacctgtgcagggcacgcaatgaacacaggaccagaaaactgcagccctaggaacactc
aagagctggccatttgcaagcatctctggcctccgtgcttctcactcatgtcccatgtct
tatacaggcctctgtggcacctcgcttgcctgatctcatccctagccgttaagctttctg
catgacttatcacttggggcataatgctggatacctaccattttcttagacccccatcaa
aatcctatttgagtgtacggttcggagaacctcatttatccggtaaatgtcttttactct
gctctcagggagctgaggcaggacatcctgagatacattgggagaggagatacagtttca
ataaaataataggttgggtggaggtacatgcctataatgccaccactcaggaaatggtgg
cagcttcgtgagtttgaggccaacccaagaaacatagtgaaaccctgtcagtaaataagt
aagcaagtatttgagtatctactatatgctagggctgacctggacattaggggtcatctt
ctgaacaaactagtgcttgagggaggtatttggggtttttgtttgtttaatggatctgaa
tgagttccagagactggctacacagcgatatgactgagcttaacacccctaaagcataca
gtcagaccaattagacaataaaaggtatgtatagcttaccaaataaaaaaattgtatttt
caagagagtgtctgtctgtgtagccctggctgttcttgaactcactctgtagaccaggct
ggcctggaaatccatctgcctgcctctgcctctctgcctctctgcctctctgcctctctc
tctgcctctctctgcctctctctgcccctctctgcccctctctgcccctctctgccgccc
tctgccgccctctgccttctgccctctgccctcgcctctggcctctgccctctgccctcg
ctgtggcctctggcctctgcctcttgagtgctggaatcaaaggtctgagctctgtaggtc
ttaagttccagaagaaagtaatgaagtcacccagcaggaggtgctcagggacagcacaga
cacacacccaggacactaggctcccacttcttggctttctctgagtggcaaaggccttag
gcagtgtcactccctaagagaaggggataaagagaggggctgaggtattcatcatgtgct
ccgtggatctcaagccctcaaggtaaatggggacccacctgtcctaccagctggctgacc
tgtagctttccccaccacag (Intron I ends)
aatccaagtcggaactcttggcaccggatcctctagagtcgac
                                                Bam HI Sal I
SEQ ID NO:3: TGase F-Thy:
                      Ned I
5′- ATGTCGACATATGGCCGAGGAGCTGGTCTT -3′
            Sal I Start codon
SEQ ID NO:4: TGase-5V40 R-Thy:
            SV40 poly A Tg II
5′- GCCGGCCGCCCCGACTCGAGTTACTTGTCA -3′
SEQ ID NO:5: TGSV4O F-Thy:
                            TgII SV40 poly A
5′- TGAGAAGTAACGCGAGTCGGGGCGGCCGGC -3′
SEQ ID NO:6: SV40 poly R-Thy:
                  Sal I Kpn I
5′- TAAGTCGACGGTACCTTATCGATTTTACCA -3′
                                        Stop codon
SEQ ID NO:7: SV40 Poly A sequence:
5′-TCGGGGCGGCCGGCCGCTTCGAGCAGACATGATAAGATACATTGATGAGTTTGGACA
AACCACAACTAGAATGCAGTGAAAAAAATGCTTTATTTGTGAAATTTGTGATGCTATTGC
TTTATTTGTAACCATTATAAGCTGCAATAAACAAGTTAACAACAACAATTGCATTCATTT
TATGTTTCAGGTTCAGGGGGAGGTGTGGGAGGTTTTTTAAAGCAAGTGAAACCTGTACAA
ATGTGGTAAAATCGATAAGGTACCGTCGACTTA -3′
      Stop Kpn I Sal I
```

1. A recombinant DNA molecule comprising a mouse neuron-specific Thy 1.2 promoter operably linked to a TGII encoding sequence.

2. A recombinant DNA molecule according to claim 1 wherein said sequence encoding said TGII fragment has the characteristics of genomic DNA.

3. A recombinant DNA molecule according to claim 1 wherein said sequence encoding said TGII fragment has the characteristics of cDNA.

4. A recombinant DNA molecule according to claim 1 wherein said sequence is that of SEQ ID NO: 1

5. A vector comprising recombinant DNA according to claim 1.

6. A vector comprising recombinant DNA according to claim 2.

7. A vector comprising recombinant DNA according to claim 3.

8. A vector comprising recombinant DNA according to claim 4.

9. A eukaryotic cell line comprising recombinant DNA according to claim 1.

10. A eukaryotic cell line comprising recombinant DNA according to claim 2.

11. A eukaryotic cell line comprising recombinant DNA according to claim 3.

12. A eukaryotic cell line comprising recombinant DNA according to claim 4.

13. A transgenic non-human mammal, and progeny thereof whose germ cells and somatic cell express recombinant DNA according to claim 1.

14. A transgenic non-human animal, or progeny, thereof, whose germ cells and somatic cells express recombinant DNA according to claim 2.

15. A transgenic non-human animal, or progeny thereof, whose germ cells and somatic cells express recombinant DNA according to claim 3.

16. A transgenic non-human animal, or progeny thereof, whose germ cells and somatic cells express recombinant DNA according to claim 4.

17. A transgenic non-human animal, or a progeny thereof, according to claim 12 which is a mouse.

18. A transgenic non-human animal, or a progeny thereof, according to claim 13 which is a mouse.

19. A transgenic non-human animal, or a progeny thereof, according to claim 14 which is a mouse.

20. A method for treating an animal having a disease characterized by the cross-linking of at least one Aβ peptide, tau, or both comprising administering a therapeutically effective amount of an inhibitor of TGII fragment.

21. A method for determining the ability of a compound to inhibit the expression of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression of said TGII polypeptide by said compound.

22. A method for generating data to determining the ability of a compound of interest to inhibit the expression of a TGII polypeptide comprising the steps of:
a. creating a transgenic non-human animal by stably incorporating into the embryonic stem cells of said animal the recombinant DNA of claim 1;
b. growing said embryonic stem cells into a mature transgenic non human animal;
c. administering to said transgenic non-human animal the compound of interest;
d. measuring the inhibition of expression of said TGII polypeptide by said compound;
e. using the data derived from said inhibition to synthesize compounds capable of inhibiting said TGII expression.


SEQUENCE LISTING (7 sequences)

SEQ ID NO: 1 - DNA, 2064 bp, Human/Homo Sapiens
atggccgaggagctggtcttagagaggtgtgatctggagctggagaccaatggccgagaccaccacacggccgacctgtgccgggagaagctggtggtgcgacggggccagcccttctggctgaccctgcactttgagggccgcaactaccaggccagtgtagacagtctcaccttcagtgtcgtgaccggcccagcccctagccaggaggccgggaccaaggcccgttttccactaagagatgctgtggaggagggtgactggacagccaccgtggtggaccagcaagactgcaccctctcgctgcagctcaccaccccggccaacgcccccatcggcctgtatcgcctcagcctggaggcctccactggctaccagggatccagctttgtgctgggccacttcattttgctcttcaacgcctggtgcccagcggatgctgtgtacctggactcggaagaggagcggcaggagtatgtcctcacccagcagggctttatctaccagggctcggccaagttcatcaagaacataccttggaattttgggcagtttcaagatgggatcctagacatctgcctgatccttctagatgtcaaccccaagttcctgaagaacgccggccgtgactgctcccggcgcagcagccccgtctacgtgggccgggtgggtagtggcatggtcaactgcaacgatgaccagggtgtgctgctgggacgctgggacaacaactacggggacggcgtcagccccatgtcctggatcggcagcgtggacatcctgcggcgctggaagaaccacggctgccagcgcgtcaagtatggccagtgctgggtcttcgccgccgtggcctgcacagtgctgaggtgcctaggcatccctacccgcgtcgtgaccaactacaactcggcccatgaccagaacagcaaccttctcatcgagtacttccgcaatgagtttggggagatccagggtgacaagagcgagatgatctggaacttccactgctgggtggagtcgtggatgaccaggccggacctgcagccggggtacgagggctggcaggccctggacccaacgccccaggagaagagcgaaggaacgtactgctgtggcccagttccagttcgtgccatcaaggagggcgacctgagcaccaagtacgatgcgccctttgtctttgcggaggtcaatgccgacgtggtagactggatccagcaggacgatgggtctgtgcacaaatccatcaaccgttccctgatcgttgggctgaagatcagcactaagagcgtgggccgagacgagcgggaggatatcacccacacctacaaatacccagaggggtcctcagaggagagggaggccttcacaagggcgaaccacctgaacaaactggccgagaaggaggagacagggatggccatgcggatccgtgtgggccagagcatgaacatgggcagtgactttgacgtctttgcccacatcaccaacaacaccgctgaggagtacgtctgccgcctcctgctctgtgcccgcaccgtcagctacaatgggatcttggggcccgagtgtggcaccaagtacctgctcaacctaaccctggagcctttctctgagaagagcgttcctctttgcatcctctatgagaaataccgtgactgccttacggagtccaacctcatcaaggtgcgggccctcctcgtggagccagttatcaacagctacctgctggctgagagggacctctacctggagaatccagaaatcaagatccggatccttggggagcccaagcagaaacgcaagctggtggctgaggtgtccctgcagaacccgctccctgtggccctggaaggctgcaccttcactgtggagggggccggcctgactgaggagcagaagacggtggagatcccagaccccgtggaggcaggggaggaagttaaggtgagaatggacctcgtgccgctccacatgggcctccacaagctggtggtgaacttcgagagcgacaagctgaaggctgtgaagggcttccggaatgtcatcattggccccgcctaa

SEQ ID NO: 2 - DNA, 4151 bp, mouse/mus musculus
gaattcagagaccgggaaccaaactagcctttaaaaaagataagtacaggagccagcaagatggctcagtgggtaaaggtgcctaccagcaagcctgacagctgagttcagtccccacgaactacgtggtaggagaggaccaaccaactctggaaatctgttctgcaaacacatgctcacacacacacacacaaatagtataaacaattttaaatttcatttaaaaataatttgtaaacaaaatcattagcacaggttttagaaagagcctcttggtgacatcaagttgatgctgtagatggggtatcattcctgaggacccaaaaccgggtctcagcctttccccattctgagagttctctcttttctcagccactagctgaagagtagagtggctcagcactgggctcttgagttcccaagtcctacaactggtcagcctgactactaaccagccatgaagaaacaaggagtggatgggctgagtctgctgggatgggagtggagttagtaagtggccatggatgtaatgaccccagcaatgctggctagaaggcatgcctcctttccttgtctggagacggaacgggagggatcatcttgtactcacagaagggagaacattctagctggttgggccaaaatgtgcaagttcacctggaggtggtggtgcatgcttttaactccagtactcaggaggcagggccaggtggatctctgtgagttcaagaccagcctgcactatggagagagttttgggacagccagagttacacagaaaaatcctggtggaaaatctgaaagaaagagagaaagaaagaaagaaagaaaggaagaaagaaagaaagagtggcaggcaggcaggcaggaggaaggaaggaaggaaggaaggaaggaaggaaggaaggaaggaaggaaggaaggaaggaaggaaaataggtgcgacttcaagatccggagttacaagcagaatgcactgtttccctaacagggccaagtgttttgagtaactgaaggtgggcatgatgcctgggaagcagaaacaagccaggcagatgcaccccttgcctttgcttccgaagggctgcagtagcatggaaaagatggaaaacaaccaatccattccctttgctgatataacaggctccaaagccaaaacctgtcactggaggctcaagagcagatctccagccaagaggcaaaggaatgggggaagctggagggcctccctctggttatccaggcttctgaaggttcaagcaaagaaagggttacaaccttaaaaggagagcgtcccggggtatgggtagaagactgctccaccccgacccccagggtccctaaccgtcttttccctgggcgagtcagcccaatcacaggactgagagtgcctctttagtagcagcaagccacttcggacacccaaatggaacacctccagtcagccctcgccgaccaccccaccccctccatccttttccctcagcctccgattggctgaatctagagtccctccctgctcccccctctctccccacccctggtgaaaactgcgggcttcagcgctgggtgcagcaactggaggcgttggcgcaccaggaggaggctgcagctaggggagtccaggtgagagcaggccgacgggagggacccgcacatgcaaggaccgccgcagggcgaggatgcaagccttccccagctacagttttgggaaaggataccagggcgctcctatatgggggcgcgggaactggggaaagaaggtgctcccaggtcgaggtgggagaggaaggcagtgcggggtcacgggctttctccctgctaacggacgctttcgaagagtgggtgccggaggagaaccatgaggaaggacatcaaggacatcaaggacagcctttggtccccaagctcaaatcgctttagtggtgcgaatagagggaggaggtgggtggcaaactggagggagcccccagcgggtgacctcgtggctggctgggtgcggggcaccgcaggtaagaaaaccgcaatgttgcgggaggggactgggtggcaggcgcgggggaggggaaagctagaaaggatgcgagggagcggaggggggagggagcgggagaatctcaactggtagaggaaagttaaaatgaggaaatagcatcagggtggggttagccaagccgggcctcagggaaaggcggcaaagtttgtctgggtgtgggcttaggtgggctgggtatgagattcggggcgccgaaaacactgctgcgcctctgccaaatcacgctacccctgtatctagttctgccaggcttctccagccccagccccaattcttttcttagtgttccttccctcccctgaatctcaagcccacactccctcctccataacccactgttatcaaatctaagtcatttgccacccaacaaccatcagcgaggcggaagcagacgggaggagtttgagatcaacttgggctacatcacgagttccaggctcaccaaggcttcttaaggagaccttgtctctaaaattaattaattaattaattaatagtcccctttctctgccacagaaccttgggatctggctcctggtcgcagctccccccaccccaggctgacattcactgccatagcccatccggaaatcctagtctatttccccatggatcttgaactgcagagagaatggcagagtggcccgccctgtgcaaaggatgttcctagcctaggtggagctcgcgaactcgcagactgtgcctctcttgggcaaggacaggctagacagcctgccggtgtgttgagctagggcactgtggggaaggcagagaacctgtgcagggcacgcaatgaacacaggaccagaaaactgcagccctaggaacactcaagagctggccatttgcaagcatctctggcctccgtgcttctcactcatgtcccatgtcttatacaggcctctgtggcacctcgcttgcctgatctcatccctagccgttaagctttctgcatgacttatcacttggggcataatgctggatacctaccattttcttagacccccatcaaaatcctatttgagtgtacggttcggagaacctcatttatccggtaaatgtcttttactctgctctcagggagctgaggcaggacatcctgagatacattgggagaggagatacagtttcaataaaataataggttgggtggaggtacatgcctataatgccaccactcaggaaatggtggcagcttcgtgagtttgaggccaacccaagaaacatagtgaaaccctgtcagtaaataagtaagcaagtatttgagtatctactatatgctagggctgacctggacattaggggtcatcttctgaacaaactagtgcttgagggaggtatttggggtttttgtttgtttaatggatctgaatgagttccagagactggctacacagcgatatgactgagcttaacacccctaaagcatacagtcagaccaattagacaataaaaggtatgtatagcttaccaaataaaaaaattgtattttcaagagagtgtctgtctgtgtagccctggctgttcttgaactcactctgtagaccaggctggcctggaaatccatctgcctgcctctgcctctctgcctctctgcctctctgcctctctctctgcctctctctgcctctctctgcccctctctgcccctctctgcccctctctgccgccctctgccgccctctgccttctgccctctgccctcgcctctggcctctgccctctgccctcgctgtggcctctggcctctgcctcttgagtgctggaatcaaaggtctgagctctgtaggtcttaagttccagaagaaagtaatgaagtcacccagcaggaggtgctcagggacagcacagacacacacccaggacactaggctcccacttcttggctttctctgagtggcaaaggccttaggcagtgtcactccctaagagaaggggataaagagaggggctgaggtattcatcatgtgctccgtggatctcaagccctcaaggtaaatggggacccacctgtcctaccagctggctgacctgtagctttccccaccacagaatccaagtcggaactcttggcaccggatcctctagagtcgac

SEQ ID NO: 3 - DNA, 30 bp, Artificial Sequence
  Artificial Sequence primers/oligonucleotides
atgtcgacatatggccgaggagctggtctt

SEQ ID NO: 4 - DNA, 30 bp, Artificial Sequence
  Artificial Sequence primers/oligonucleotides
gccggccgccccgactcgagttacttgtca

SEQ ID NO: 5 - DNA, 30 bp, Artificial Sequence
  Artificial Sequence primers/oligonucleotides
tgacaagtaacgcgagtcggggcggccggc

SEQ ID NO: 6 - DNA, 30 bp, Artificial Sequence
  Artificial Sequence primers/oligonucleotides
taagtcgacggtaccttatcgattttacca

SEQ ID NO: 7 - DNA, 270 bp, Artificial Sequence
  Artificial Sequence primers/oligonucleotides
tcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtgaaacctgtacaaatgtggtaaaatcgataaggtaccgtcgactta